(19) United States
(12) Patent Application Publication
(10) Pub. No.: US 2006/0240117 A1
(43) Pub. Date: Oct. 26, 2006

(54) Snake powder extract for treatment of cancer

(76) Inventor: Pedro J. Lecca, Chevy Chase, MD (US)

Correspondence Address: DELPHINE M. JAMES, 2656 SOUTH LOOP WEST #170, HOUSTON, TX, 77054, US

(21) Appl. No.: 11/336,630

(22) Filed: Jan. 20, 2006

Related U.S. Application Data
(63) Continuation-in-part of application No. 10/306,958, filed on Dec. 2, 2002, now abandoned.

(51) Int. Cl. A61K 35/58

(52) U.S. Cl. 424/542; 435/375

(57) ABSTRACT

The present invention relates to a pharmaceutical composition comprising snake powder that is derived from the Tzabcan “Crotalus durissus” rattlesnake. The snake powder is prepared by continuously baking the body of the rattlesnake until it completely dehydrates. Then, the dehydrated body is pulverized into a fine granular powder. The present invention included an in vitro method of inhibiting cancer cell growth utilizing the snake powder exhibited. Accordingly, the snake powder can be applied for the development of drugs which are effective for the treatment of various types of cancers.

### BACKGROUND

The present invention relates to a composition matter that exhibits anti-cancer properties, more particularly, snake powder which was derived from pulverized dehydrated snake body of the Tzabcan “Crotalus durissus” rattlesnake.

Cancer is one of the major causes of death in the United States. The use of snakes to treat diseases has been utilized for over a decade. More particularly, there are several patents utilizing the snake venoms from various species of snakes. One example of the use of snake venom to treat cancer is described in U.S. Pat. No. 5,565,431. The snake venom is extracted to create an immune enhancer to help cure cancer through Immuno-therapy. Immuno-therapy, also called biologic therapy, uses the body's own immune system to fight cancer cells or protect the body from the side effects. Immuno-therapy relies on antibodies, which are naturally occurring proteins in the body dedicated to defending the body against invasion by foreign substances. In Immuno-therapy, the antibodies are used to attack the tumor cells directly. The present invention

Venom from different snake species contains compounds of various biological activities (1). Snake venom, is a mixture of proteins with different structures (1). Scientists have isolated compounds with known biological activity from snake venom. Such activities include local and systemic hemorrhage (2); tissue damage and impaired muscle regeneration (3), intracranial hemorrhage (4); cardiovascular shock (5); decreased oxygen utilization by tissues and increased plasma glucose and lactate concentrations (6), disturbances in atria-ventricular conduction and reduction in amplitude and duration of action potential (6); hypotension in man (6); interaction with blood coagulation system, endothelial cells and platelets; analgesic activity (7), blood coagulation (8), proteolytic, phosphodiesterase, hyaluronidase, thrombin-like or kallikrein-like, phospholipase and protease activities (9), bradykinin-potentiating activity and an angiotensin-converting enzyme (10), platelet inhibition aggregation (11), platelet coagglutinin (12). But the antitumor activity of some of these compounds is of great importance, considering the need for agents with anti-tumor effect.

There is no information (as revealed by our literature search) on the use of snake powder in the treatment of cancer in the clinic in the practice of modern medicine. However literature is replete with the use of the components of snake venom for cancer chemotherapy. Snake venom contains anti-tumor compounds. Albolabrin, eristostatin, echistatin, contortrostatin, salmosin and jararhagin are compounds obtained from snake venom with anti-tumor effect. They contain an Arg-Gly-Asp [RGD] sequence, have a high cysteine content and are of low molecular weight. The presence of an RGD sequence implies that these compounds recognize the integrin receptors (α3β1, α5β1, αvβ1, αv3, αvβ5, αvβ6 and αvβ8) which play a big role in tumor metastasis. Studies have shown that these compounds are antagonists on fibrinogen receptor associated with glycoprotein IIb/IIa complex and also inhibit platelet aggregation (13). Their ability to act as an antagonist make this group of compounds a potential target in drug discovery for potential anti-metastastic drugs. They inhibit adhesion of B16-F10 melanoma cells to extra-cellular matrices (fibronectin, fibrinogen, vitronectin and collagen type 1). These class of compounds are grouped into either disintegrin or metalloproteinase compounds.

A phase I clinical trial study was performed to evaluate the maximum tolerated dose, safety profile and pharmacokinetic data with VRCTC-310, a natural product derived from purified snake venom fractions, with phospholipase A2 activity and inhibitory effects against human and murine tumor cell lines. Fifteen patients with refractory malignancies were entered after providing written informed consent. Maximum tolerated dose was 0.017 mg/kg and was recommended for Phase II clinical trial studies (14). A multidisciplinary study has been carried out on the inhibitory effect of a snake venom contortrostatin (a protein) isolated from Agkistrodon contortrix (southern copperhead) venom on breast cancer progression. Contortrostatin, injected daily at 5 microgram per mouse to MDA-MB-435 tumor masses in an orthotopic xenograft nude mouse model inhibited growth of tumor by 74%. It was shown that contortrostatin is not cytotoxic to cancer cells, and does not inhibit proliferation of the breast cancer cells in vitro. Its mechanism of action is by inhibiting angiogenesis induced by breast cancer, as shown by immunohistochemical quantitation of the vascular endothelial cells in tumor tissue removed from the nude mice (15).

A snake venom disintegrin was found to inhibit human ovarian cancer dissemination and angiogenesis in an orthotopic nude mouse model (16). Rhodostomin, purified from the snake venom of Calloselasma rhodostoma was found to inhibit angiogenesis elicited by basic fibroblast growth facto and suppressed tumor growth of subcurtaneously inoculated B16F10 solid tumor, leading to a prolonged survival of the rhodostomin—treated C57BU6 mice (17). Salmosin, a snake venom-derived compound that antagonizes platelet aggregation, was found to significantly inhibit bovine capillary endothelial cell proliferation induced by fibroblast growth factor but had no effect on normal growth of cell. Both the meatastatic-tumor growth and solid tumor growth that developed in mice were effectively suppressed by salmosin treatment (18).

Sharma et. al. (19) reported that consumption of rattlesnake capsule (desiccated rattlesnake meat formulated in hard gelatin capsules) and powders for medicinal purposes is of high prevalence among Mexican-American individuals living on the border with Mexico. The patients appeared to seek this folk remedy as a cure for a variety of maladies such cancer, acquired immunodeficiency syndrome (AIDS), diabetes and diabetes, which are chronic medical illnesses (19,20). Other reports have indicated the use of snake capsules (21 ,22).

However, Sarizonae Arizona (Arizona hinshawin) is commonly found in cold-blooded animals such as rattlesnakes and turtles; warm-blooded animals including mammals and birds, can also carry the infection, which typically involves the gastrointestinal and genitourinary systems (19, 23). Consequently, in a survey, 82% of 22 Mexican-American patients who were culture positive for S Arizona reported ingesting snake powder capsules. Further, in early 1987, two hospitals in Los Angels County, Calif., reported four cases of S. Arizona infection and all patients gave a history of ingesting rattlesnake capsules prior to onset of illness (20).

### SUMMARY

With the present invention a systematic study was done to identify various fractions, using bioactivity-guided assay, responsible for the anticancer property of snake property. From several years of research studying non-traditional methods in treating various diseases such as headaches, fever, stomach ailments, etc it was found that in Latin America the Pipiles people (descendants from the Aztecs and Mayas) are using “Cascabel” or “Rattlesnake”. From several experiments conducted, it was found that the Tzabcan “Crotalus durissus” was very effective and strong immune system enhancer. It has been used in Latin American for various diseases including cancer with possible use in diabetes, ulcers, severe burns, infections, and gangrene. The natives prepare the concoctions in several ways. They liquefied the snake and used it topically; they dried the snake and used it.

### BRIEF DESCRIPTION OF DRAWINGS

FIG. 1 illustrates the effect of Snake Extract on Tumor Cell Apoptosis using DMSO extract.

FIG. 2 illustrates the induction of cytotoxicity by snake extract using PBS solution.

FIG. 3 illustrates the induction of cytotoxicity by snake extract using DMSO solution.

FIG. 4 illustrates the induction of cytotoxicity by snake extract using PBS solution.

FIG. 5 illustrates the induction of cytotoxicity by snake extract using Ethanol solution.

FIG. 6 illustrates the effect of snake extract on tumor cell proliferation using DMSO extract.

FIG. 7 illustrates the effect of snake extract on tumor cell proliferation using Ethanol extract.

FIG. 8 illustrates the effect of snake extract on tumor cell proliferation using PBS extract.

Table 1 illustrates the results of utilizing the snake powder in three persons infected with terminal cancer.

### DETAILED SPECIFICATION

The present invention is based on the applicant's research of the practices by Latin American tribes utilizing snake methodology for healing various illnesses. It is the applicant's contention that capturing the rattlesnake in question and reducing it to a powder component, will allow it to be used for cancer treatment. The key to this concept is in using the entire body of the animal, not just the venom, which has been used in prior studies.

The applicant found evidence to show that the introduction of snake extract in a powdered form has produced some positive cellular activity associated with testing done with the extract in Ethanol, PBS, and DMSO solutions. Further testing has shown that when this powdered form was administered in controlled amounts to three people with various forms of terminal cancer, marked improvement was shown over the span of three months to one and one-half years.

The process of the present invention first requires that the captured rattlesnake's Tzabcan “Crotalus durissus” head and tail be removed from the rest of the body. The remaining mid-section of the body of the rattlesnake is then baked in an oven at a temperature of at least 320 degrees. Heat is a known means of sterilization in the art. The remaining mid-section is baked until it is dehydrated. After the mid-section is fully dehydrated, it is then pulverized into a powder form. The Tzabcan “Crotalus durissus” species of the rattlesnake has shown to be the most effective against the treatment of cancer.

Initially, the powdered form was tested in Ethanol, PBS, and DMSO solutions as described below:

1. Induction of cytotoxicity by snake extract in Ethanol solution, PBS solution and DMSO solution

2. Effect of snake extract on tumor cell proliferation in Ethanol solution, PBS and DMSO solution

3. Effect of snake extract on tumor cell apoptosis in DMSO solution

The following cell lines were utilized in the tests listed above:
Leukemia/Lymphoma: K562, Raji, Jurkatt, CEM;
Breast Carcinoma MCF-7, MDA-MB-231, MD-MB-468;
Prostate Carcinoma LNCaP, PC-3;
Colorectal Carcinoma: HT-29, HCT-116;
Pancreatic Carcinoma: PANC-1;
Gliobastaoma: GL10-1.

Laboratory analysis utilizing the PBS solution, ethanol solution, DMSO solution on the above listed cell lines has been found to interfere with the effect of the positive benefits of the snake powder in inhibiting the growth of cancer cells for the treatment of cancer. However, even with the diminished effects brought on by the use of experimental solution mediums, the positive effects of the snake powder were not totally destroyed. Each experiment is analyzed below.

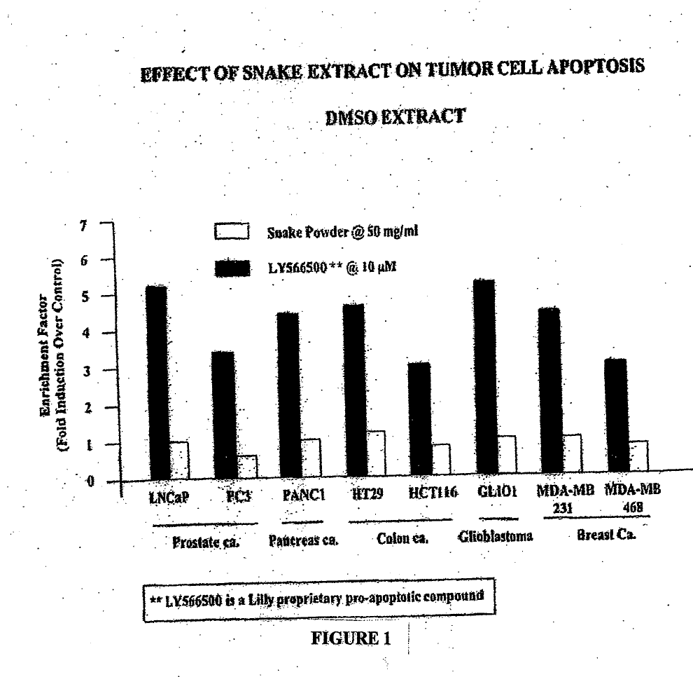

In reference to FIG. 1, the effect of the snake extract on tumor cell apoptosis with dimethysulfoxide (DMSO) solution is analyzed. The methodology used to analyze apoptosis was nucleosomal fragmentation. With this methodology, the experiments quantified the histone-complexed DNA fragmentation (i.e. mono nucleosomes and oligonucleosomes) out of the cytoplasm of cells after the induction of apoptosis by the stimuli. The stimuli used in this experiment were snake powder and the LY566500.

The cell lines investigated in this experiment were as follows:
Breast Carcinoma MCF-7, MDA-MB-231, MD-MB-468;
Prostate Carcinoma LNCaP, PC-3;
Colorectal Carcinoma: HT-29, HCT-116;
Pancreatic Carcinoma: PANC-1;
Gliobastaoma: GL10-1.

The extract was prepared by the following process:
4 g of powder was extracted with DSMO
the extract was evaporated utilizing a speed vacuum centrifuge
when dried the extract was resuspended in fresh DMSO at 1 g/ml.
a sample of 50 mg/ml was utilized in the experiment

Apoptosis has been defined as a form of programmed cell death and it plays a major role during development as a major mechanism for the precise regulation of cell numbers and as a defense mechanism to remove unwanted and potentially dangerous cells that have been infected by viruses and tumor cells. In all the cancer cell lines investigated, as shown in FIG. 1, LY566500 (Lilly proprietary pro-apoptotic compound) appeared to be better able to cause cell death (apoptosis) than the snake powder. However, the snake powder did show some marked activity of cell death (apoptosis) in each cell line investigated.

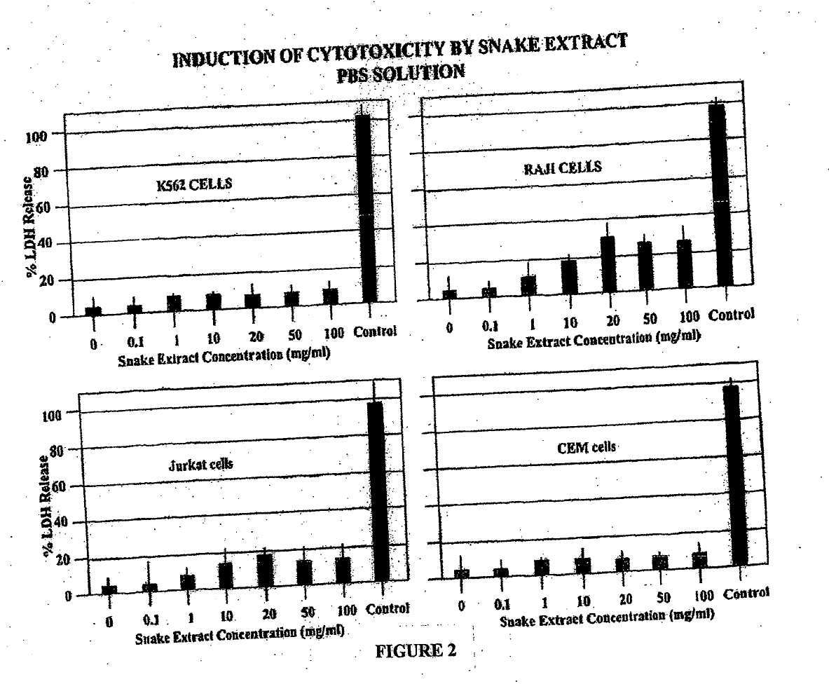

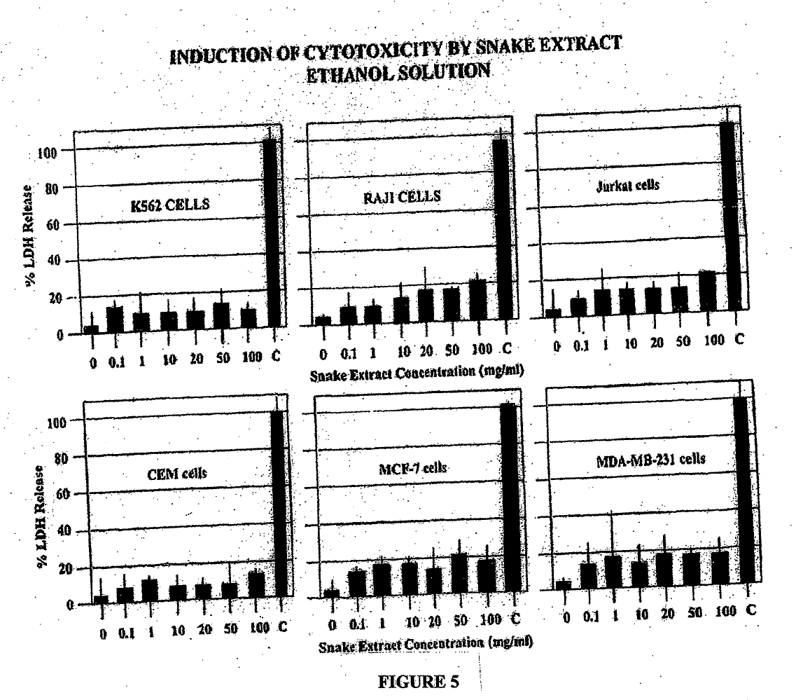

In reference to FIGS. 2, 3, and 4 the induction of cytotoxicity by snake extract utilizing the PBS solution and DMSO solution are respectively analyzed. In reference to FIG. 5, the induction of cytotoxicity by snake extract utilizing the ethanol solution is analyzed. The methodology used to analyze the induction of cytotoxicity was Lactate dehydrogenase (LDH) assay. With this methodology, target cancer cells are incubated with a cytotoxic agent (i.e. snake powder and Camptothecin). During the incubation period, cytoplasmic LDH is released into the culture supernatant due to plasma membrane damage. The LDH activity in the culture supernatant is measured by substrated reaction and quantified by ELISA.

The DMSO and ETHANOL extract was prepared by the following process:
4 g of powder was extracted with each
the extract was evaporated utilizing a speed vacuum centrifuge
when dried the extract was re-suspended in fresh solution at 1 g/ml.
the concentration of extract used ranges from 0.1 to 100 mg/ml

The PBS solution was prepared as follows:
dissolve 1 g/ml of powder within PBS solution
the concentration of extract used ranges from 0.1 to 100 mg/ml

The following Leukemia/Lymphoma cell lines were used in the experiment:
K562—chronic myelogenous leukemia,
Raji—B-cell lymphoma,
Jurkatt—T-cell leukemia,
CEM—acute T Lymphoblastic leukemia

It is known that small amounts of enzymes are present in the blood circulation at all times. Lactate dehydrogenase (LDH) is one of these enzymes. LDH catalyzes the conversion of lactate and pyruvate. Consequently, LDH represents a group of enzymes that are present in almost all metabolizing cells and about five individual isoenzymes make up the total LDH serum level. When tissue damage occurs, the LDH is released into the blood in larger quantities. In fact, the serum levels of the enzymes are often used as an aid in the diagnosis of certain diseases. Thus, the release of LDH is analyzed

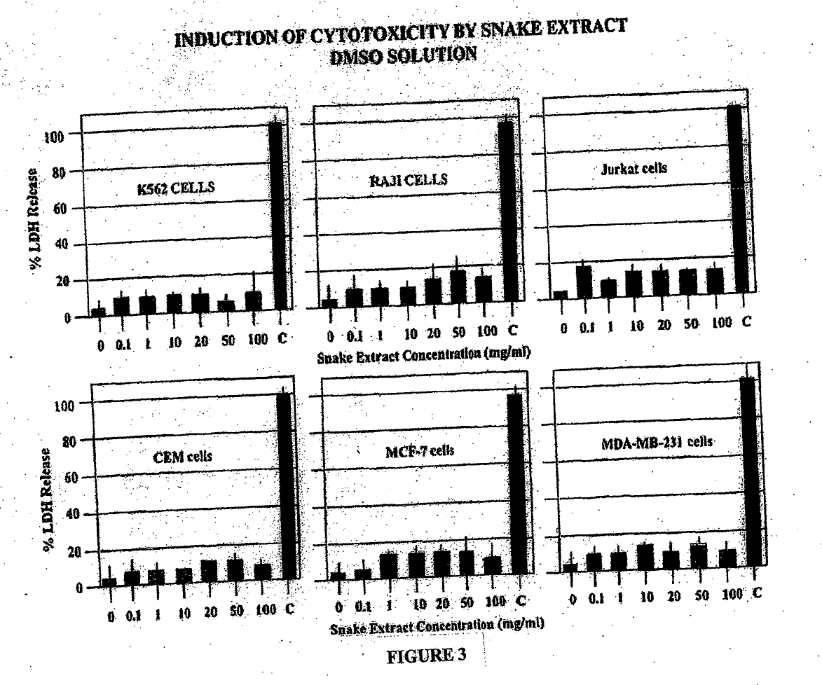

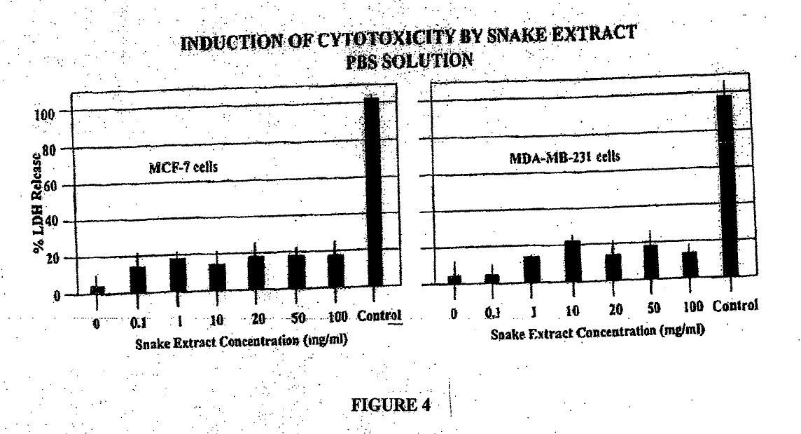

Referring to FIG. 2, the snake extract prepared using PBS buffer, at all concentrations investigated, was less injurious to different types of cell lines because it caused less percent release of LDH. As shown in FIG. 3, a similar pattern was observed when cytotoxicity was induced utilizing the snake extract in the DMSO solution. As shown in FIG. 4, a similar pattern was observed when cytotoxicity was induced utilizing the snake extract in the PBS solution against additional cell lines. As shown in FIG. 5, a similar pattern was observed when cytotoxicity was induced utilizing the snake extract in the ethanol solution.

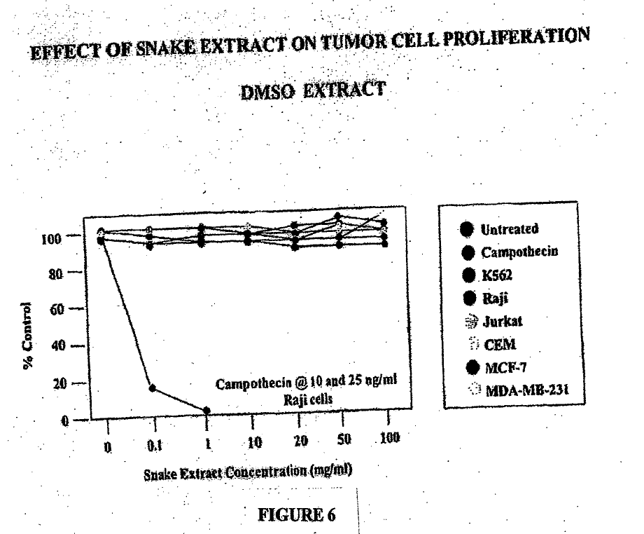

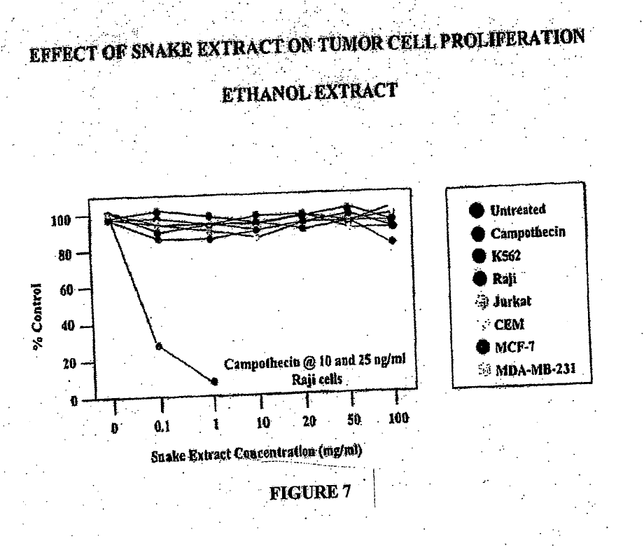

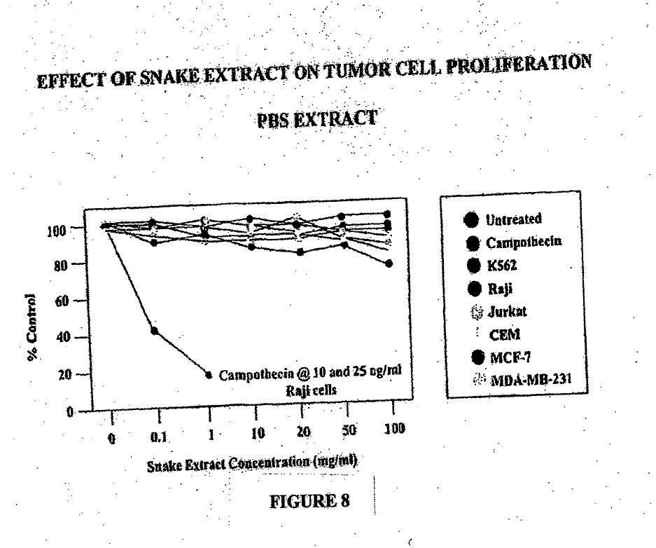

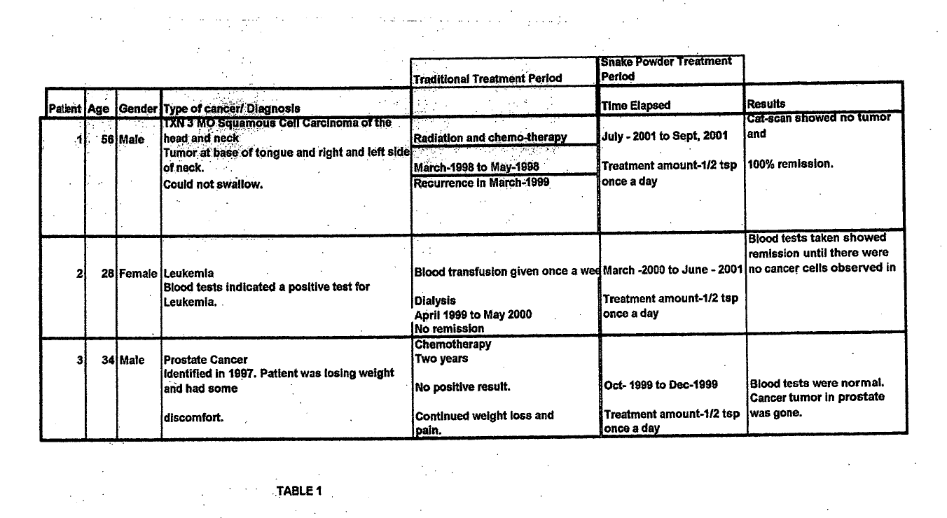

Referring to FIGS. 6, 7, and 8 the effect of snake extract on tumor cell proliferation utilizing the DMSO, ethanol, and PBS solutions, is respectively analyzed. The methodology used to analyze tumor cell proliferation was oxygen biosensor. With this methodology culture plates are coated with an oxygen sensitive fluorescent compound embedded in a gas-permeable and hydrophobic matrix permanently attached to the bottom of a multi-well plate. The amount of fluorescence correlates directly to the rate of oxygen consumption in the well, providing a signal that can be directly correlated to cell growth.

Camptothecin is known to be active against tumors that are normally considered chemorefractory such as colorectal and lung tumors. Camptothecin inhibits topoisomerase I, an enzyme critical to the growth of tumor cells. Further, Camptothecin can also cause single strand breaks in DNA.

Referring to FIGS. 6, 7, and 8, Camptothecin is more effective at inhibiting cell proliferation of different cell lines than the snake powder extract. However, in each case analyzed the snake extract showed some marked activity when compared with untreated cell lines. The snake extract was more effective in the PBS solution than the DMSO and ethanol solution.

In each case analyzed in FIGS. 1-8, the snake powder extract produced some positive cellular activity associated with testing done in the ethanol, PBS, and DMSO solutions. It was concluded that the solvents might interfere with activity of the snake powder extract, thereby causing reduced activity.

Further experiments were done utilizing laboratory rats to analyze the anti-free radical and immune function properties of the pure snake powder. Pure snake powder was injected into the blood stream of laboratory rats. Results showed that the activity of sodium dismutase enzyme (SOD) in the erythrocytes of rats increased. The T-lymphocyte transformation in the peripheral blood of the rats increased. It was concluded that the pure snake powder has anti-free radical action and can increase immune function in experimental animals.

Further testing using the undiluted powder by three persons having various forms of terminal cancer was conducted. The results of these tests showed a significant increase in the ability of each person's immune system to naturally inhibit the growth of cancer tumor cells. The results of the tests are shown in Table 1.

Referring to the first cancer patient in Table 1, this patient took the snake powder after completing chemotherapy and radiation therapy. The patient is Male, was born on Jul. 25, 1942, and had head and neck cancer. Patient 1 was diagnosed with head and neck cancer and had received chemotherapy, (drugs) and radiation treatments with no positive results. When the first patient started taking the snake powder on Jul. 21, 2001, he could not swallow. On Jul. 28, 2001, swallowing improved. After taking the snake powder for six weeks with no other medication or treatment, his condition improved. A CT-Scan of the neck was performed which showed no lymphadenopathy, no abnormal mass, or no enhancement within the neck area Additionally, Patient 1 had blood cultures that indicated the cancer was not spreading and was reduced. By Sep. 3, 2001, this Patient swallowing was normal.

In reference to the second cancer patient in Table 1, the patient was a 28-year-old female, who was diagnosed in March of 2000 through blood tests that indicated a positive test for Leukemia. Blood transfusions were given once a week for 14 months. Additionally, during the months of April 2000 and May 2000, the patient was treated with dialysis. The Leukemia remained with no satisfactory remission at that time.

The second patient began taking the snake powder in March 2001 and continued until June 2001. After her treatment period, a blood test was taken which showed remission of her cancer. Another blood test was performed in August 2002, which again showed no cancer cells in the blood. In reference to the third cancer patient, the patient was a 34 year old male with prostate cancer identified in 1997. He was treated with chemotherapy for two years with no positive result, with continued weight loss and pain. The patient started taking the snake powder in late October 1999 until December 1999. After the treatment period, his blood test was normal, and the cancer tumor in the prostate area was eradicated. He was able to return to work.

TABLE 1 Snake Powder Treatment Traditional Period Patient Age Gender Type of cancer/Diagnosis Treatment Period Time Elapsed Results 156 Male TXN 3 MO Squamous Cell Carcinoma of the Radiation and July 2001 to Cat-scan showed no head and neck chemo-therapy September 2001 tumor and Tumor at base of tongue and right and left side March 1998 to May 1998 Treatment amount-100% remission of neck. Recurrence in March 1999 ½ tsp Could not swallow. once a day Blood transfusion given 2 28 Female Leukemia once a week March 2000 to Blood tests taken Blood tests indicated a positive test for Dialysis June 2001 showed remission Leukemia April 1999 to Treatment amount—until there were no May 2000 ½ tsp cancer cells No remission once a day observed in 3 34 Male Prostate Cancer Chemotherapy October 1999 to Blood tests were Identified in 1997. Patient was losing weight Two years December 1999 normal. Cancer and had some No positive result. Treatment amount—tumor in prostate discomfort. Continued weight loss and ½ tsp was gone pain once a day

Further experiments were done to determined the cancer growth inhibiting properties of the snake powder through the induction of cytotoxicity.

### EXPERIMENT I

### Induction of Cytotoxicity Analyzed Using Nauplii

The purpose of this experiment is to determine preliminary indication for anti-cancer property in the snake powder by investigating the biological activity of the snake powder. The brine shrimp egg assay is a simple and inexpensive test for this purpose.

Materials Utilized

Powdered snake material, methanol, distilled water, dichloromethane, ethylacetate, butanol, sodium bicarbonate.

The snake extract was prepared utilizing the steps below:

(a) Methanolic Extraction and Solvent Partitioning

20 g of pulverized dried snake powder was extracted by maceration with 200 ml of methanol at room temperature for 2 hours. The extract was filtered and concentrated in vacuo using a rotary evaporator [I]. 0.89 g of the methanol extract was reconstituted in 50% aqueous methanol. The aqueous methanolic fraction was partitioned between hexane and water. The aqueous layer was further extracted successively with dichloromethane[B], ethylacetate [C] and butanol [D]. The remaining aqueous layer was basified with sodium bicarbonate to pH 8 and then extracted with dichloromethane [E]. Extracts A, B, C, D, E were then concentrated in vacuo. The material residue was re-extracted in methanol overnight, filtered and concentrated in vacuo [IB]

(b) Aqueous Extraction

3.5 g of pulverized dried material was extracted by maceration with 35 ml distilled water at room temperature for 2 hrs. The extract was filtered, centrifuged and dried by lypholization [II]. The material residue, was re-extracted in water overnight, filtered, centrifuged and lyophilized [IIB]

Result

| Extract | Code | Weight (g) | Yield(%) |
|---|---|---|---|
| Methanol | I | .92 | 4.6 |
| Methanol | IB | .5 | |
| Hexane | A | .07 | 7.9 (% I) |
| Dichloromethane | B | .05 | 5.6 (% I) |
| Ethylacetate | C | .002 | .23 (% I) |
| Butanol | D | .14 | 15.7 (% I) |
| Dichloromethane | E | .01 | 1.1 (% I) |
| Water | II | .276 | 7.9 |
| Water | IIB | .15 | |

These extracts were stored in the refrigerator.

AIM-3 Bioassay studies were conducted on the partitioned extracts.

Brine Shrimp Lethality Assay

The eggs of brine shrimp, Airtime salina (Leach), are used in monitoring bioactive compounds. The eggs are readily available in pet shops at low cost and remain viable for years in the dry state. The brine shrimp assay has advantages of being rapid (24 hrs), inexpensive, and simple. No aseptic techniques are required. It easily utilizes a large number of organisms for statistical validation and requires no special equipment and a relatively small amount of sample. Furthermore, it does not require animal serum as is needed for cytotoxicities.

Materials Used

Artemia salina eggs, methylsulphoxide, distilled water

Experimental Method

The assay was carried out with Artemia salina eggs. 50 mg of Artemia salina eggs in a beaker containing sea salt (6 g), yeast (0.9 mg) and distilled water (150 ml) were incubated at 27-28° C. for 24 hrs. The newly hatched nauplii were concentrated. From this volume, aliquots of 50 μl (approximately 18-20 nauplii) were pipette directly into a 24 well plates containing sea water (sw), −ve control (DMSO+sw), +ve control (emetine) or different concentrations of extracts (62.5-500 μg/ml). Assay was carried out in triplicate. The plates were sealed and incubated at 27-28° C. for 24 h. At the end of the 24-h incubation period, the content of each well was pipette into a watch glass individually. Survival was assessed by scoring the number of dead nauplii using a microscope. Once counts of dead nauplii had been taken, 0.5 ml of methanol was added to kill all remaining nauplii. The contents of each well were then recorded and result tabulated as (X/Y; where X number dead and Y=number of dead and alive).

Result

| | Concentration (% mortality | | | | |
|---|---|---|---|---|---|
| Sample | 62.5 | 125 | 250 | 500 | Extract |
| A | 5 | 6.5 | 18 | 64.3 | Hexane |
| B | 0 | 18.4 | 7.4 | 16.7 | Dichloromethane |
| C | 2.4 | 3.3 | 4.8 | 10.5 | Ethylacetate |
| D | 0 | 1.6 | 9.8 | 6.3 | Butanol |
| E | 0 | 9.7 | 0 | 19.6 | Dichloromethane |
| F | 11.1 | 0 | 14.8 | 16.4 | Butanol |
| G | 3.2 | 20.8 | 18.5 | 3.5 | Methanol |
| | | | | | (crude) |
| H | 0 | 8.8 | 21.2 | 12.1 | Crystal from |
| | | | | | methanol |
| Water extract | 13.3 | 19.4 | 47.5 | 41.2 | Water |
| | | | | | (freeze |
| | | | | | dried) |
| Water extract | 16.7 | 37.1 | 24.1 | 21.7 | Second |
| [2nd extract | | | | | extraction |
| Emetine(+ve | 58.2 | 100 | 100 | 100 | |
| control) | | | | | |
| −ve control | 0 | 0 | 0 | 0 | |

Conclusion

Cytotoxicity was observed at high concentration for all extracts of the snake powder. In the order of activity, hexane extract was the most active followed by the water extract. The activity of extracts E, F, G and H increased with concentration, while extract D showed a decrease in activity with increase in concentration.

### EXPERIMENT II

### Cell Viability Assay

The Objective of this experiment is to screen a crude aqueous snake extract on Human HL-60 Leukemia (CCL240) cell lines. In vitro cell viability was measured using the tetrazolium dye (MTT) assay.

Materials Used

Doxorubicin powder and MTT were purchased from Sigma Inc, Methyl sulfoxide was obtained from Aldrich, and Sodium dodecyl sulfate was obtained from Chemika, USA. Phosphate buffer saline [PBS] was obtained from Invitrogen, Iscove's modified Dulbecco's medium was obtained from ATCC. The freeze-dried aqueous extract of a snake material was prepared in this laboratory.

Preparation of Standard Solution

Stock solution of the aqueous snake extract (2 mg/ml), Doxorubicin (125 μg/ml) were dissolved in pre-warmed PBS [37° C.] and made up to required concentration with culture medium. Working solution of the snake extract, 1000, 500,250,125 μg/ml and doxorubicin 15.65, 31.25, 62.5, and 125 μg/ml were taken from the stock solution by adjusting the volume of the multichannel pipette.

MTT [5 mg/ml] was prepared in PBS. The pH was adjusted to between 6.0-6.4 with 0.1M HCl. Sodium dodecyl sulfate (10%) was prepared in methyl sulfoxide

Tumor Cell Lines

Human Leukemia [HL-60] cell lines [CCL 240] was obtained from ATCC [USA]. The cell lines were grown in Isovec's Modified Dulbecco's Medium with 20% heat inactivated fetal calf serum, 1-% penicillin/streptomycin. Cells were incubated in a humidified atmosphere of 5% CO2/95% air at densities that promoted exponential proliferation

Cell Viability Assay

n vitro cell viability was measured using the tetrazolium dye (MTT) assay. 100 μl of ATCC 240 Human leukemia cell line containing 160 cells were seeded in 96-multiwell plates and pre-cultured for 24 hours before drug treatment. Various concentrations of crude extract [125-1000 μg/ml] and doxorubicin [15.65-125 μg/ml] were added to different wells in sixplicates. After 72 hours of incubation, 20 μl of MTT solution (5 mg/ml) was added to each well and plates were incubated at 37° C. for 4 hours. 25 μl of 10% SDS. in methylsulfoxide was added to each well to solubilized any MTT product formed with viable cells. Absorbance was measured using an automated microplate reader at a wavelength of 560 nm each representing the average of six wells. To investigate if the color of doxorubicin, interferes with absorption, four controls were set up [control 1-200 μl of isovec's solution; control 2-100 μl of doxorubicin+100 μl PBS; control 3-200 μl of doxorubicin solution; control 4-100 μl of cell culture+100 μl of culture medium]. The cell survival was expressed as a percentage of the control 4. The experiments were repeated four times.

A MTT assay for anticancer activity of the aqueous extract of the snake powder on PC-3 prostate cancer cells was observed after 72 Hours.

[image]

B MTT assay for anticancer activity of the methanol extract of the snake powder on PC-3 prostate cancer cells was observed after 72 hours.

[image]

C MTT assay for anticancer activity of the aqueous extract of the snake powder on Human HI-60 Leukemia Cell Lines [Crl 2258] was observed after 72 Hours.

[image]

From our results, we may conclude that the mechanism underlying the antitumor activity of the aqueous snake extract is not by direct cytototoxic effect. In support of this our reasoning, is our previous result on the brine shrimp lethality tests, where the aqueous snake extract, did not show any toxicity on the brine shrimp cells.

Conclusion: In-vitro cell viability, as measured by the tetrazolium dye (MTT) assay, indicates that the extracts have anticancer property at high concentrations as indicated in TABLE 2 and TABLE 4.

In conclusion, from the above experiments, it is my contention that the ingredients in the snake powder enhance the immune system to prevent the cancer cells from growing and allowing the natural mechanisms of body system (i.e. the white blood cell), to attack and to kill the cancer cells.

What is claimed is:

1. An in vitro method of inhibiting growth cells of a cancer or a tumor:
providing a fine granular snake powder prepared by the steps of:
(a) removing a head and a tail from a body of a rattlesnake;
(b) continuously, baking the remaining mid-section of the body of the rattlesnake at a sufficient temperature to dehydrate the mid-section; and
(c) pulverizing the mid-section into the fine granular snake powder;
mixing a sufficient amount of the fine granular snake powder into an experimental solution medium; and
exposing the cells to the fine granular snake powder
wherein exposing the cells to the fine granular snake powder inhibits the growth thereof.

2. The method of claim 1 wherein the experimental solution medium is ethanol,

3. The method of claim 1 wherein the sufficient amount ranges between 0.1 to 100 milligrams per milliliter of experimental solution medium.

4. The method of claim 1 wherein the sufficient temperature is at least 320 degrees.

5. The method of claim 1 wherein the cells are selected from the group consisting of Leukemia/Lymphoma, Breast Carcinoma, Prostate Carcinoma, Colorectal Carcinoma, Pancreatic Carcinoma and Gliobastaoma.

6. A pharmaceutical composition produced by the method of claim 1.
